(19) United States
(12) Patent Application Publication
(10) Pub. No.: US 2018/0303943 A1
(43) Pub. Date: Oct. 25, 2018

(54) Dosage form comprising two-dimensional structural elements

(71) Applicant: Aron H. Blaesi, (US)

(72) Inventors: Aron H. Blaesi, Cambridge, MA (US); Nannaji Saka, Cambridge, MA (US)

(73) Assignee: Aron H. Blaesi, Cambridge, MA (US)

(21) Appl. No.: 15/964,063

(22) Filed: Apr. 26, 2018

Related U.S. Application Data
(63) Continuation-in-part of application No. PCT/US16/58935, filed on Oct. 26, 2016.
Application No. PCT/US16/58935 is a continuation-in-part of application No. 15/482,776, filed on Apr. 9, 2017.

(51) Int. Cl. A61K 47/34

(52) U.S. Cl. CPC A61K 47/34

(57) ABSTRACT

The most prevalent pharmaceutical dosage forms at present, the oral-delivery tablets, are granular solids. An inherent limitation of such granular solids for drug release applications is the unpredictability of the microstructure. As a result, the drug release rate and other properties are difficult to control, and their range is also limited. Presented herein, therefore, is a solid dosage form with predictable microstructure and properties. The dosage form includes a drug-containing solid comprising a three dimensional structural framework of one or more two-dimensional structural elements.

### CROSS-REFERENCE TO RELATED APPLICATIONS

This application is a continuation-in-part of, and incorporates herein by reference in its entirety, the International Application No. PCT/US16/58935 filed on Oct. 26, 2016 and titled “Solid Dosage Form for Immediate Drug Release and Apparatus and Method for Manufacture thereof”. This application is also a continuation-in-part of, and incorporates herein by reference in its entirety, the commonly owned U.S. application Ser. No. 15/482,776 filed on Apr. 9, 2017 and titled “Fibrous dosage form”.

This application is related to, and incorporates herein by reference in its entirety, the commonly owned U.S. application Ser. No. 14/907,891 filed on Jan. 27, 2016 and titled “Melt-Processed Polymeric Cellular Dosage Form”. This application is also related to, and incorporates herein by reference in its entirety, the International Application No. PCT/US17/47703 filed on Aug. 19, 2017 and titled “Method and apparatus for the manufacture of fibrous dosage forms”. Further, this application is related to, and incorporates herein by reference in its entirety, the International Application No. PCT/US17/41609 filed on Jul. 11, 2017 and titled “Method and apparatus for the manufacture of cellular solids”.

### FIELD OF THE INVENTION

This invention relates generally to microstructures and compositions for drug release. In certain embodiments, the invention relates to solid dosage forms comprising at least one two-dimensional structural element.

### BACKGROUND OF THE INVENTION

The most prevalent pharmaceutical dosage forms at present, the oral immediate-release tablets, are porous solids consisting of compacted drug and excipient powders. Although powder processing is extensively used in the manufacture of oral dosage forms, an inherent limitation of compacted powders is the non-deterministic porosity. As a result, the dosage form microstructure and properties (e.g., the drug content, drug release rate, etc.) are difficult to control tightly, and their range is also limited.

To overcome such limitations, therefore, in the commonly owned U.S. patent application Ser. No. 14/907,891, the commonly owned U.S. patent application Ser. No. 15/482,776, and the publications in J. Control. Release, 220 (2015) 397-405; Eur. J. Pharm. Biopharm, 103 (2016) 210-218; Int. J. Pharm. 509 (2016) 444-453; Chem. Eng. J. 320 (2017) 549-560; Mater. Sci. Eng. C 80 (2017) 715-727; and Mater. Sci. Eng. C 84 (2018) 218-229, the present inventors (Blaesi and Saka) have introduced cellular and fibrous dosage forms. These dosage forms comprise solid frameworks of a drug-excipient composite (or a solid solution) and gas-filled cells or voids. It was shown that both the microstructure and the drug release rate are predictable and precisely controllable. The release rate was predominantly determined by the physico-chemical properties of the excipient, the connectivity of the void space, the cell size (or inter-fiber spacing in the case of fibrous dosage forms), and the wall thickness (or fiber radius).

A related structural framework that enables predictable properties, a greater range of properties, and faster and more economical development and manufacture of dosage forms at reproducible quality, among others, comprises two-dimensional structural elements. Therefore, in this disclosure, microstructures and compositions of dosage forms comprising two-dimensional structural elements are presented. It may be noted that the terms “two-dimensional structural elements”, “two-dimensional elements”, and “elements” are used interchangeably herein.

### SUMMARY OF THE INVENTION

In a first aspect, the present invention provides a pharmaceutical dosage form comprising a drug-containing solid having an outer surface and an internal structure contiguous with and terminating at said outer surface; said internal structure comprising a three dimensional structural framework of one or more two-dimensional elements; said two-dimensional elements comprising at least one active ingredient and at least one excipient; said two-dimensional elements further comprising segments separated and spaced from adjoining segments by free spacings; and the free spacings defining one or more free spaces in said drug-containing solid.

In certain embodiments, the internal structure further comprises one or more zero-dimensional elements.

In certain embodiments, the internal structure further comprises one or more one-dimensional elements.

In certain embodiments, the one or more 2-dimensional elements comprise an average thickness no greater than 2.5 mm.

In certain embodiments, the free spacing between the segments is so that the percolation time of physiological/body fluid into one or more interconnected free spaces of the dosage form is no greater than 900 seconds under physiological conditions.

In certain embodiments, the effective free spacing between the segments across the one or more free spaces on average is greater than 0.1 μm.

In certain embodiments, the position of at least one two-dimensional element or at least one segment in the internal structure is precisely controlled.

In certain embodiments, the three dimensional framework of one or more two-dimensional elements comprises an ordered structure.

In certain embodiments, the thickness of at least one two-dimensional element is precisely controlled.

In certain embodiments, at least one excipient is wettable by a physiological/body fluid under physiological conditions.

In certain embodiments, at least one excipient is soluble in a physiological/body fluid and comprises a solubility greater than 0.1 g/l in said physiological/body fluid under physiological conditions.

In certain embodiments, dissolved molecules of the soluble excipient comprise a diffusivity greater than 0.2×10⁻¹² m²/s in a physiological/body fluid under physiological conditions.

In certain embodiments, at least one excipient is absorptive of a physiological/body fluid, and wherein rate of penetration of the physiological/body fluid into a two-dimensional element or said absorptive excipient under physiological conditions is greater than the average thickness of said two-dimensional element divided by 3600 seconds.

In certain embodiments, at least one excipient is absorptive of a physiological/body fluid, and wherein an effective diffusivity of physiological/body fluid in a two-dimensional element or said absorptive excipient is greater than 0.5×10⁻¹¹ m²/s under physiological conditions.

In certain embodiments, at least one excipient transitions from solid to a fluidic or gel consistency solution upon contact with a volume of physiological/body fluid equal to the volume of the one or more free spaces of the drug-containing solid, said solution having a viscosity less than 500 Pa·s under physiological conditions.

In certain embodiments, at least one excipient is selected from the group comprising polyethylene glycol (PEG), polyethylene oxide, polyvinylpyrrolidone (PVP), PEG-PVP copolymer, poloxamer, lauroyl macrogol-32 glycerides, polyvinylalcohol (PVA), PEG-PVA copolymer, polylactic acid, polyvinylacetate phthalate, polymethacrylates (e.g., poly(methacrylic acid, ethyl acrylate) 1:1, or butylmethacrylat-(2-dimethylaminoethyl)methacrylat-methylmathacrylat-copolymer), gelatin, cellulose or cellulose derivatives (e.g., microcrystalline cellulose, hydroxypropyl cellulose, hydroxyethyl cellulose, methyl cellulose, hydroxypropyl methyl ether cellulose, or hydroxypropyl methylcellulose), starch, polylactide-co-glycolide, polyvinyl caprolactam-polyvinyl acetate-polyethylene glycol graft copolymer, lactose, starch derivatives (e.g., pregelatinized starch or sodium starch glycolate), chitosan, pectin, polyols (e.g., lactitol, maltitol, mannitol, isomalt), acrylic acid crosslinked with allyl sucrose or allyl pentaerythritol (e.g., carbopol), and polyacrylic acid.

In certain embodiments, a free space is filled with a matter selected from the group comprising gas, liquid, or solid, or combinations thereof, and wherein said matter is partially or entirely removed upon contact with a physiological/body fluid under physiological conditions.

In certain embodiments, the gas comprises at least one of air, nitrogen, CO₂, argon, or oxygen.

In certain embodiments, the free spaces are interconnected.

In certain embodiments, less than twelve walls must be ruptured to obtain an interconnected cluster of free space from the outer surface of the drug-containing solid to any point in the internal structure.

In a second aspect, the present invention provides a a pharmaceutical dosage form comprising a drug-containing solid having an outer surface and an internal structure contiguous with and terminating at said outer surface; said internal structure comprising a three dimensional structural framework of one or more two-dimensional elements; said two-dimensional elements comprising at least one active ingredient and at least one excipient; said two-dimensional elements further comprising segments separated and spaced from adjoining segments by free spacings; and the free spacings defining one or more free spaces in said drug-containing solid; wherein the one or more two-dimensional elements comprise an average thickness no greater than 2.5 mm; the effective free spacing between the segments across the one or more free spaces on average is between 0.1 μm and 2 mm; and at least one dimension of the dosage form is greater than 1 mm.

In a third aspect, the present invention provides a a pharmaceutical dosage form comprising a drug-containing solid having an outer surface and an internal structure contiguous with and terminating at said outer surface; said internal structure comprising a three dimensional structural framework of one or more two-dimensional elements; said two-dimensional elements comprising at least one active ingredient and at least one excipient; said two-dimensional elements further comprising segments separated and spaced from adjoining segments by free spacings; and the free spacings defining one or more free spaces in said drug-containing solid; wherein the one or more two-dimensional elements comprise an average thickness no greater than 2.5 mm; the effective free spacing between the segments across the one or more free spaces on average is between 0.1 μm and 2 mm; at least one dimension of the dosage form is greater than 1 mm; and

at least one excipient comprises a solubility greater than 0.1 g/l in a physiological/body fluid under physiological conditions or at least one excipient is absorptive of a physiological/body fluid, and wherein rate of penetration of the physiological/body fluid into a two-dimensional element or an absorptive excipient under physiological conditions is greater than average thickness of the two-dimensional elements divided by 3600 seconds.

Elements of embodiments described with respect to one aspect of the invention can be applied with respect to another aspect. By way of example but not by way of limitation, certain embodiments of the claims described with respect to the first aspect can include features of the claims described with respect to the second or third aspect, and vice versa.

This invention may be better understood by reference to the accompanying drawings, attention being called to the fact that the drawings are primarily for illustration, and should not be regarded as limiting.

### BRIEF DESCRIPTION OF THE DRAWINGS

The objects, embodiments, features, and advantages of the present invention are more fully understood when considered in conjunction with the following accompanying drawings:

FIG. 1 shows non-limiting schematic diagrams of the microstructure of dosage forms comprising a three dimensional structural framework of two-dimensional elements according to this invention;

FIG. 2 presents schematic diagrams of microstructures of additional embodiments of solid dosage forms according to this invention;

FIG. 3 schematically shows microstructure and disintegration of a single two-dimensional structural element by interdiffusion of polymeric excipient molecules and dissolution fluid in both stagnant and stirred media;

FIG. 4 schematically presents the dissolution/disintegration of a dosage form structure in a stagnant dissolution fluid;

FIG. 5 illustrates schematics of fluid flow around and through a dosage form structure in a stirred dissolution fluid;

FIG. 6 presents a non-limiting example of percolation of dissolution medium into an interconnected free space;

FIG. 7 illustrates a schematic of the contact angle of a fluid droplet on a surface;

FIG. 8 depicts a non-limiting schematic diagram of the microstructure of solid dosage forms according to this invention to illustrate the number of walls that must be ruptured to obtain an interconnected cluster of free space that extends from the outer surface of the drug-containing solid to a point in the interior;

FIG. 9 presents three two-dimensional elements of different thickness;

FIG. 10 presents a dosage form comprising at least two drug-containing solids;

FIG. 11 is a schematic of a non-limiting process to produce the dosage forms disclosed herein;

FIG. 12 depicts a scanning electron micrograph of a dosage forms according to this invention;

FIG. 13 displays the results of the fraction of drug dissolved versus time of a dosage form according to this invention.

### DEFINITIONS

In order for the present disclosure to be more readily understood, certain terms are first defined below. Additional definitions for the following terms and other terms are set forth throughout the specification.

In this application, the use of “or” means “and/or” unless stated otherwise. As used in this application, the term “comprise” and variations of the term, such as “comprising” and “comprises,” are not intended to exclude other additives, components, integers or steps. As used in this application, the terms “about” and “approximately” are used as equivalents. Any numerals used in this application with or without about/approximately are meant to cover any normal fluctuations appreciated by one of ordinary skill in the relevant art.

Moreover, in the disclosure herein, the terms “one or more active ingredients” and “drug” are used interchangeably. As used herein, an “active ingredient” or “active agent” refers to an agent whose presence or level correlates with elevated level or activity of a target, as compared with that observed absent the agent (or with the agent at a different level). In some embodiments, an active ingredient is one whose presence or level correlates with a target level or activity that is comparable to or greater than a particular reference level or activity (e.g., that observed under appropriate reference conditions, such as presence of a known active agent, e.g., a positive control).

Furthermore, in the context of the invention herein, a three dimensional structural framework of one or more two-dimensional structural elements comprises a structure (e.g., an assembly or an assemblage or an arrangement of one or more two-dimensional structural elements) that extends over a length, width, and thickness greater than 200 μm. This includes, but is not limited to structures of one or more two-dimensional structural elements that extend over a length, width, and thickness greater than 500 μm, or greater than 700 μm, or greater than 1 mm, or greater than 1.25 mm, or greater than 1.5 mm, or greater than 2 mm.

As used herein, the terms “two-dimensional structural element”, “two-dimensional element”, “two-dimensional elements”, “2D-elements”, “one or more two-dimensional elements”, “one or more drug-containing two-dimensional elements”, “drug-containing two-dimensional elements”, and “element” or “elements” are used interchangeably. They are understood as the solid, drug-containing structural elements (or building blocks) that make up the three dimensional structural framework (e.g., the dosage form structure). A two-dimensional structural element is referred to as having a length and width much greater than its thickness. In the present disclosure, the length and width of a two-dimensional sructural element are greater than 2 times its thickness. This includes, but is not limited to a length and with greater than 3 times its thickness, or greater than 4 times its thickness, or greater than 5 times its thickness. An example of a two-dimensional element is a “sheet”.

Moreover, as used herein, the term “segment” refers to a fraction of a two-dimensional element along the length or width of said element.

As used herein, a one-dimensional structural element is referred to as having a length much greater than its width or thickness. In the present disclosure, the length of a one-dimensional structural element is greater than 2 times its width and thickness. An example of such an element is a “fiber”. It may be noted that the terms “1-dimensional element”, “one-dimensional structural element”, “one-dimensional element”, “1D-element”, and “element” are used interchangeably herein. A zero-dimensional structural element is referred to as having a length and width of the order of its thickness. In the present disclosure, the length and width of a zero-dimensional structural element are no greater than 2 times its thickness. Furthermore, the thickness of a zero-dimensional element is less than 2.5 mm. Examples of such zero-dimensional elements are “particles” or “beads” and include polyhedra, spheroids, ellipsoids, or clusters thereof. It may be noted that the terms “0-dimensional element”, “zero-dimensional structural element”, “zero-dimensional element”, “0D-element”, and “element” are used interchangeably herein.

In some embodiments herein, the term “element” may refer to a two dimensional element, or a one-dimensional element, or a zero-dimensional element.

Finally, as used herein, the terms “dissolution medium”, “physiological/body fluid”, “dissolution fluid”, “medium”, “fluid”, and “penetrant” are used interchangeably. They are understood as any fluid produced by or contained in a human body under physiological conditions, or any fluid that resembles a fluid produced by or contained in a human body under physiological conditions. Examples include, but are not limited to: water, saliva, stomach fluid, gastrointestinal fluid, saline, etc. at a temperature of 37° C. and a pH value adjusted to the specific physiological condition.

### DETAILED DESCRIPTION OF THE INVENTION

### Dosage Form Structures

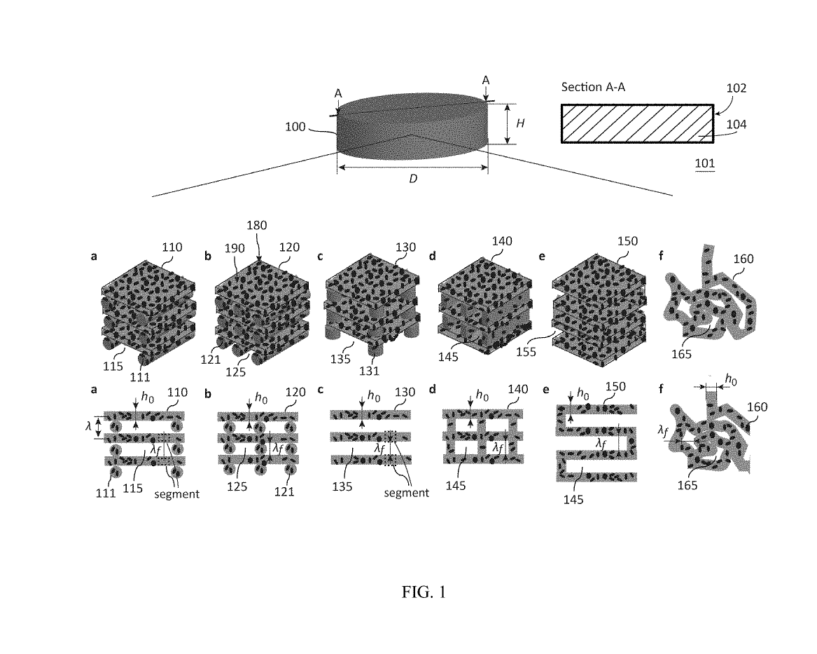

FIG. 1 presents non-limiting examples of pharmaceutical dosage forms 100 comprising a drug-containing solid 101 having an outer surface 102 and an internal structure 104 contiguous with and terminating at said outer surface 102. The internal structure 104 comprises a three dimensional structural framework of one or more two-dimensional elements 110, 120, 130, 140, 150, 160. The two-dimensional elements further comprise segments separated and spaced from adjoining segments by free spacings, λ_{f}, which define one or more free spaces 115, 125, 135, 145, 155, 165 in the drug-containing solid 101. The two-dimensional elements 110, 120, 130, 140, 150, 160 may be oriented (e.g., arranged or structured) in a variety of ways, ranging from random (e.g., disordered) to partially regular (e.g., partially ordered) to regular (e.g., ordered or not random).

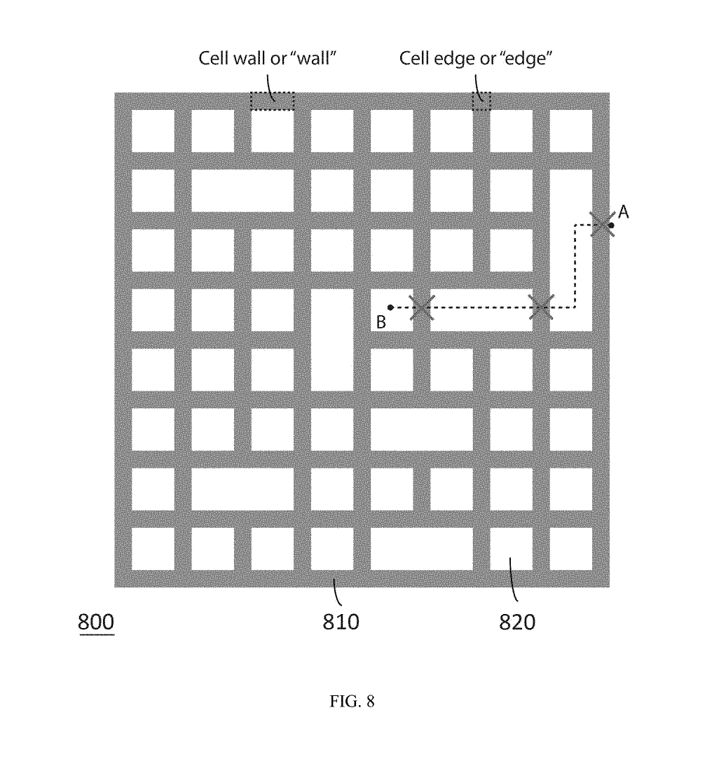

FIG. 1a shows a dosage form 100 with parallel arrangement (e.g. a three dimensional structural framework with parallel arrangement) of two-dimensional elements 110 with rectangular cross section. In between the two-dimensional elements 110 are layers of one-dimensional elements 111 to separate segments from adjoining segments by free spacings, λ_{f}. The free spacings define one or more free spaces 115 in the dosage form 100. This arrangement (or structure, or three dimensional structural framework) is ordered and provides control of two structural variables essential for tailoring the properties of the dosage form 100: the thickness of the two-dimensional element 110 or sheet, h, (or the average thickness, h₀) and the spacing between the segments, λ (or alternatively the free spacing, λ_{f}). The free spaces 115 between the segments are intrinsically connected to the outer surface 102 of the dosage form 100 in this arrangement. Thus by the commonly used terminology to describe cellular structures (see, e.g., M. F. Ashby, “The mechanical properties of cellular solids”, Metall. Trans. A, 14A (1983) 1755-1769; L. J. Gibson, M. F. Ashby, “Cellular solids: structure and properties”, second edition, Cambridge University Press, 1999; and the example of FIG. 8 of the specification herein), the two-dimensional elements 110 essentially form the walls of cells. Some of the cell walls are removed to connect the free spaces 115 to the outer surface 102 of the dosage form 100.

Other non-limiting three dimensional structural frameworks of one or more two-dimensional elements are presented in FIGS. 1b-1d. FIG. 1b shows a three dimensional structural framework of elements 120 as in FIG. 1a but with the one-dimensional elements more closely together. In FIG. 1c the three dimensional structural framework of one or more elements 130 further comprises zero-dimensional elements 131 instead of one-dimensional elements 111, 121 to separate segments from adjoining segments by free spacings, λ_{f}. FIG. 1d is an non-limiting example of a three dimensional structural framework with interpenetrating two-dimensional elements 140. FIG. 1d shows a non-limiting example of a continuous two-dimensional element 150 that makes up the three-dimensional structural framework. FIG. 1e is a structure with random or almost random arrangement/assembly of one or more two-dimensional elements 160 (e.g. a structure that is disordered).

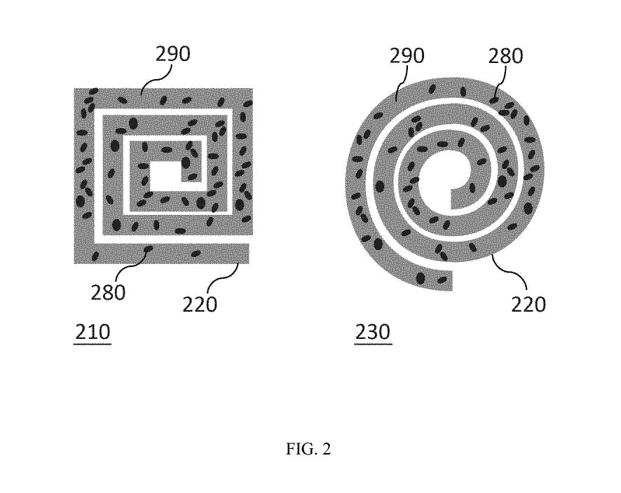

Yet other non-limiting examples of three dimensional structural frameworks of one or more two-dimensional elements are shown in FIG. 2, which presents a top view of two-dimensional elements 220 in a plane forming a rectangular structure 210, as well as a top view of two-dimensional elements 220 in a plane forming a circular (or elliptical) structure 230.

More examples of how the two-dimensional elements may be structured, arranged, or assembled would be obvious to a person of ordinary skill in the art. All of them are within the spirit and scope of this invention.

### Compositions and Material Structures of Two-Dimensional Elements

The two-dimensional elements 110, 120, 130, 140, 150, 160, 220 typically consist of one or more active ingredients 180, 280 (also referred to here as “drug”), and in most cases also one or more excipients 190, 290 (also referred to here as “excipient”). If a two-dimensional element consists of at least one active ingredient and at least one excipient, the drug and excipient may be structured in the two-dimensional element in an ordered or “partially or completely disordered” manner. Moreover, the structural features of the drug or the excipient in the two-dimensional elements may comprise any shape or geometry. By way of example but not by way of limitation, this includes particles, beads, polygons, ellipsoids, cubes, tubes, rods, sheets, etc., or combinations thereof. The features may have a size at the molecular-, nano-, micro-, meso-, or macro-scale. Thus, drug may be molecularly dissolved in excipient, excipient may be molecularly dissolved in drug, drug may be dispersed as nano- or micro-particles in an excipient, and so on.

More such examples of compositions and material structures of two-dimensional elements would be obvious to a person of ordinary skill in the art. All of them are within the scope of this invention.

### Drug Release from Two-Dimensional Elements

If the composition of a two-dimensional element consists of drug only, or if the drug is interconnected in the material structure of the two-dimensional element, the drug may be in direct contact with dissolution fluid upon immersion of the two-dimensional element in a medium. Thus, in some embodiments, the drug may be released from the two-dimensional element by dissolution of drug into the medium.

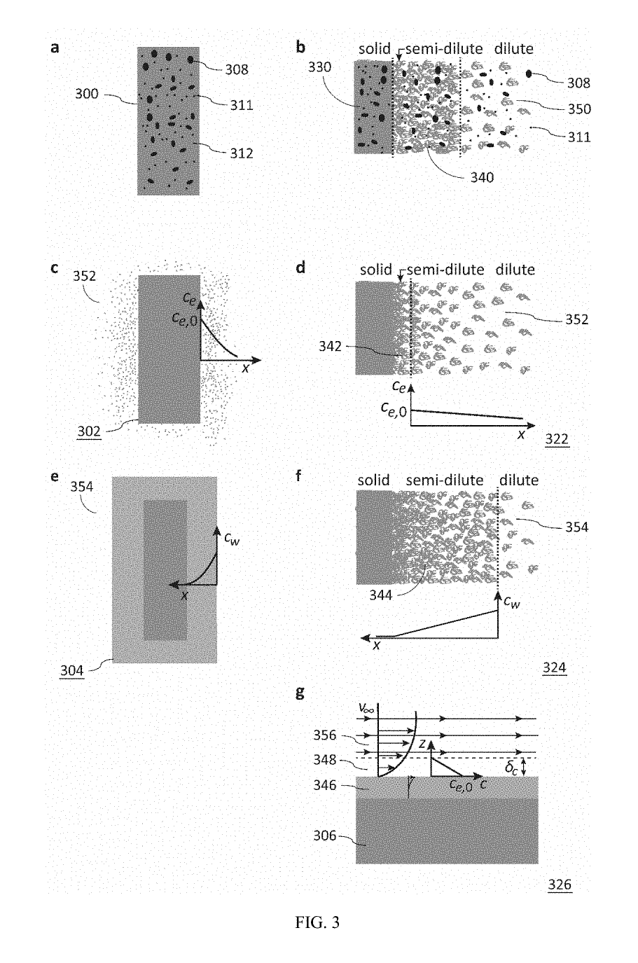

If the material structure of a two-dimensional element 300, however, comprises one or more discontinuous clusters of at least one drug particle 308 or at least one drug molecule 309 surrounded by a solid excipient 312 as shown in FIG. 3a, erosion or swelling of the excipient 312 is a prerequisite for drug release from the two-dimensional element 300. Two non-limiting examples of how drug may be released from such two-dimensional elements 300 are presented below.

In the first non-limiting example, the excipient comprises an erodible polymer. Thus, as soon as the two-dimensional element 300 is brought in contact with dissolution medium, the medium diffuses into the excipient. The penetrant molecules (e.g., the dissolution fluid that diffused into the solid excipient) may then induce the solid excipient to swell (e.g., to increase in volume) and to transition from a solid to a fluidic or gel consistency solution. Subsequently, the polymer molecules from the gel consistency solution may diffuse or erode into the dissolution medium. The drug (e.g., a drug molecule or a drug particle) may be released from the two-dimensional element 300 as soon as the surrounding excipient has converted to dissolved molecules or a gel with polymer concentration smaller than the “interfacial concentration”.

The “interfacial concentration” is referred to in this application as the polymer concentration which separates the “solid” and “liquid” regions. For a typical polymer that erodes into a dissolution fluid, the interface is diffuse, and thus the interfacial concentration is difficult to determine precisely. As schematically shown in FIG. 3b, the diffuse interface may extend over a layer 340 of non-negligible but finite thickness. It may be considered a semi-dilute gel consistency solution between the entangled, concentrated, and viscous polymer 330 (i.e., the “solid” or “semi-solid”) and the dilute, low-viscosity dissolution medium 350 (i.e., the “liquid”). Thus, typically, the concentration of an eroding polymer in the semi-dilute interfacial layer 340 (e.g., the “interfacial concentration”) is of the order of the disentanglement concentration, cₚ*, of said polymer in a dissolution medium. In some embodiments, however, if the rate at which polymer molecules at the interface are disentangled is small, the interfacial concentration may be substantially smaller than cₚ*. (For further information related to polymer disentanglement, see e.g., P. G. De Gennes, “Scaling concepts in polymer physics”, fifth ed., Cornell University Press, 1996; or M. Doi, S. F. Edwards, “The theory of polymer dynamics”, Oxford University Press, 1986).

In the second non-limiting example, the excipient comprises an absorptive or swellable polymer. Thus, upon immersion of the two-dimensional element in a dissolution fluid, the fluid diffuses into the solid polymeric excipient. The penetrant molecules (e.g., the dissolution fluid that diffused into the solid excipient) may then convert part or all of the solid drug enclosed in the polymeric excipient to dissolved drug molecules. The mobility of drug molecules may be greater in the penetrated polymeric excipient than in the excipient without penetrant. Thus the drug molecules embedded in the penetrated excipient may diffuse to the dissolution medium swiftly, and drug may be released within the specific time requirements.

More examples of drug release from two-dimensional elements would be obvious to a person of ordinary skill in the art. All of them are within the scope of this invention.

### Modeling Disintegration and Drug Release

The following examples present ways by which the drug release and disintegration behavior of two-dimensional elements and dosage forms comprising two-dimensional elements may be modeled. The models will enable one of skill in the art to more readily understand the properties and advantages of the dosage forms disclosed. The models and examples are presented by way of illustration, and are not meant to be limiting in any way.

(a) Erosion of Two-Dimensional Elements by Diffusion without Convection

FIGS. 3c and 3d show a non-limiting example of a polymeric element with rectangular cross section 302 and its interface 322 after immersion in an unstirred, infinite dissolution medium 352. The excipient polymer molecules are assumed to diffuse away from the interface faster than the dissolution medium diffuses into the element. Thus after a short wait after immersion, the thickness of the diffuse, semi-dilute layer 342 is (and remains) thin compared with the element thickness or the thickness of the dilute region 352. The dissolution rate (or the disintegration rate) of the element 302 may thus be described by the diffusion of excipient molecules from the element interface into the dilute medium. The initial rate of erosion of the element 302 may be approximated by:

$$\frac{1}{2} \frac{dh}{dt} = - \frac{{j}_{e}}{{ρ}_{e}} \approx - \frac{{c}_{e , 0}}{{ρ}_{e}} \sqrt{\frac{{D}_{e}}{π t}} \qquad ( 1 )$$

Integrating gives

$$h ( t ) = {h}_{0} - \frac{2 {c}_{e , 0}}{{ρ}_{e}} \sqrt{\frac{4 {D}_{e} t}{π}} \qquad ( 2 )$$

where h(t) is the element's thickness as a function of time, h₀ the initial thickness of the element, jₑ the flux of the eroding excipient polymer, ρₑ the density of the solid excipient, c_{e,0} the interfacial concentration of the excipient polymer, and Dₑ the diffusivity of an excipient molecule in the dissolution medium.

By way of example but not by way of limitation, if h₀=250 μm, c_{e,0}=163 kg/m³, ρₑ=1150 kg/m³, Dₑ=1.09×10⁻¹⁰ m²/s, the element thickness decreases to about 170 μm after the time t=h₀²/Dₑ=9.6 mins. Thus about 32% of the element are dissolved or disintegrated at this time in this example. By contrast, if the element thickness is increased to 5 mm (a typical thickness of a dosage form) and the other parameters are kept the same, only about 1.6% would be eroded 9.6 minutes after immersion in a still fluid. This percentage is more than an order of magnitude smaller than the corresponding value of a thin element. The advantage of a “thin” element over a “thick” element or dosage form for achieving fast disintegration (and high drug release) rates is thus exemplified.

It would be obvious to a person of ordinary skill in the art that the model presented (and any of the following models) are readily adapted to two-dimensional elements of non-rectangular cross section. Such elements include, but are not limited to two-dimensional elements with elliptical, polygonal, or any other cross section. Furthermore, more examples of models of erosion of a single element in a still dissolution medium would be obvious to a person of ordinary skill in the art. All of them are within the scope of this invention.

(b) Diffusion of Dissolution Fluid into an Element

FIGS. 3e and 3f present another non-limiting example of a polymeric element 304 and its interfacial region 324 after immersion in a dissolution fluid 354 that is of infinite extent and stagnant (not stirred). Now it is assumed that water (or dissolution fluid) diffusion into the excipient polymer is faster than polymer diffusion into the fluid. This is opposite of the previous case. In this model, the thickness of the gel-layer 344 grows with time as dissolution fluid continues to diffuse in. Under Fickian diffusion (see, e.g., J. Crank, “The Mathematics of Diffusion”, second edition, Oxford University Press, 1975), the time taken by the dissolution fluid 354 to penetrate the element 304 (i.e., to convert it into a gel) may be estimated as:

$${t}_{pen} = \frac{{h}_{0}^{2}}{4 {D}_{eff}} \qquad ( 3 )$$

where D_{eff} is an effective diffusivity of physiological/body fluid in the polymeric element under physiological conditions. By way of example but not by way of limitation, if h₀=250 μm and D_{eff}=2×10⁻¹⁰ m²/s, by Eq. (3) tₚₑₙ=78 seconds. Conversely, if h₀ is increased to 5 mm and D_{eff} remains unchanged, tₚₑₙ increases to 520 minutes. Thus the penetration time of a “thin” element is much shorter than that of a “thick” element or a “thick” dosage form of the same composition.

More such examples of models of diffusion of dissolution fluid into a single element would be obvious to a person of ordinary skill in the art. All of them are within the scope of this invention.

### (c) Disintegration of Penetrated Elements

A penetrated element may be considered a polymeric solution (or dispersion or gel) that has a viscosity greater than the viscosity of the dissolution fluid. If the viscosity of the solution (e.g., a penetrated element, the surface of a penetrated element, etc.) is small enough, and if such external forces applied on the element as gravity, shear, or imbalances in fluid pressure are large enough, the solution may deform or break up into pieces. Thus, in some embodiments a penetrated element or a penetrated surface of an element may disintegrate and dissolve rapidly.

(d) Erosion of Element with Convection

FIG. 3g schematically shows a non-limiting example of a polymeric element that erodes in a stirred medium by convective mass transfer 326. The solid polymeric excipient 306 and the dissolution medium 356 are separated by a gelated interfacial layer 346, 348. The excipient concentration at the outer boundary of the layer is zero. It increases towards the interior and reaches the density of the solid at the inner boundary. The velocity of the dissolution medium 356 is equal to the far-filed velocity, v_{∞}, far away from the interface. It decreases towards the inner boundary of the interfacial layer, and may be considered zero when the excipient concentration exceeds a critical value. Thus, a “critical” concentration may separate the interfacial layer into a dilute, moving concentration boundary layer 348 of thickness, δ_{c}, and a concentrated, highly viscous, stagnant layer 346. A reasonable estimate or definition of the critical concentration is the concentration, c_{e,0}.

In this model, for an element that erodes from both faces by convection (e.g., in a rotating basket of a USP dissolution apparatus), the erosion rate per eroding face may be approximated by:

$$E = - \frac{1}{2} \frac{dh}{dt} = 0.62 ( \frac{{D}_{e} {c}_{e , 0}}{{ρ}_{e}} ) {( \frac{{μ}_{l}}{{D}_{e} {ρ}_{l}} )}^{\frac{1}{3}} {( \frac{{ρ}_{l} Ω}{{μ}_{l}} )}^{\frac{1}{2}} \qquad ( 4 )$$

where ρₗ is the density and μₗ the viscosity of the dissolution fluid, and Ω is the angular velocity of the rotating basket. The disintegration time of the element of initial thickness H₀ eroding from both faces is about:

$${t}_{E} = \frac{{h}_{0}}{dh / dt} \qquad ( 5 )$$

(It may be noted that in the present non-limiting example, erosion from the sides is not considered because the thickness of an element is smaller than its width or length. Furthermore, we may note that the model may be adapted if the eroding surfaces are not planar.)

By way of example but not by way of limitation, if c_{e,0}=163 kg/m³, Dₑ=1.09×10⁻¹⁰ m²/s, ρₑ=1150 kg/m³, ρₗ=1000 kg/m³, μₗ=0.001 Pa·s, Ω=5.24 rad/s, and h₀=250 μm, by Eqs. (4) and (5) the calculated 0.8×t_{E}=3.6 min. By contrast, if h₀ is increased to 5 mm, 0.8×t_{E} is 73 min.

Thus, also in this non-limiting example, the “thin” element disintegrates more than an order of magnitude faster than the “thick” element or the “thick” minimally-porous dosage form. Further details related to convective mass transfer models are given, e.g., in V. G. Levich, “Physicochemical Hydrodynamics”, Prentice-Hall, Englewood Cliffs, N J, 1962; for further details related to the USP dissolution apparatus, see, e.g., The United States Pharmacopeial Convention, USP 39-NF 34. Any more examples of models of element erosion with convection obvious to a person of ordinary skill in the art are all within the scope of this invention.

### (e) Dosage Form Disintegration in a Stagnant Medium

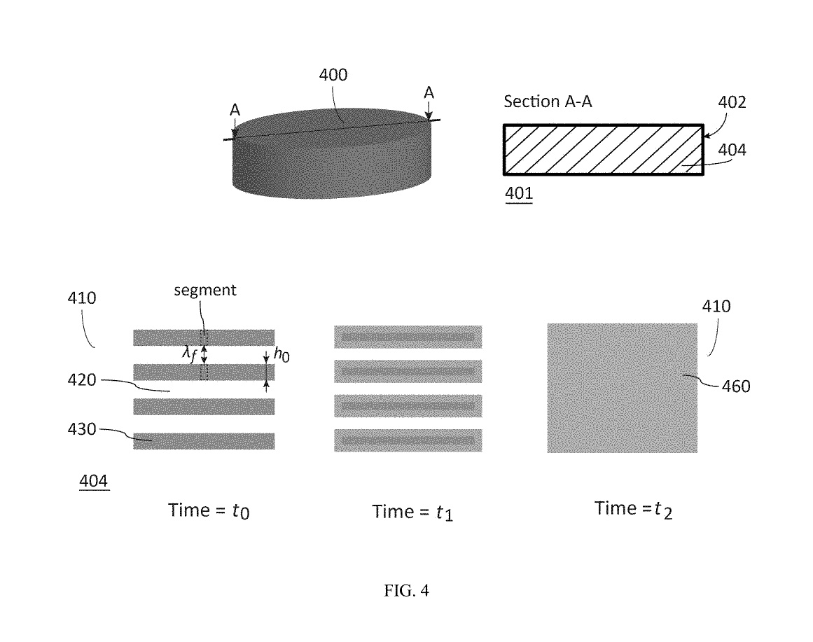

FIG. 4 presents a non-limiting example of the disintegration process of a dosage form 400 in a stagnant dissolution fluid. The dosage form 400 comprises a drug-containing solid 401 having an outer surface 402 and an internal structure 404 contiguous with and terminating at said outer surface 402. The internal structure 404 comprises a three dimensional structural framework of one or more two-dimensional elements 430. The elements 430 contain an active ingredient and a polymeric excipient that is absorptive of or soluble in (e.g., erodible by) a dissolution medium. The elements 430 further comprise segments separated and spaced from adjoining segments by free spacings, λ_{f}, which define one or more free spaces 420 in the drug-containing solid 401.

Upon immersion of the dosage form 400 in a dissolution fluid 410, the free spaces 420 may be percolated rapidly by the fluid 410 if (a) the free spaces 420 are (partially or entirely) connected to the outer surface, (b) the content of the free spaces 420 is partially or entirely removable by the dissolution fluid 410, (c) the free spacing, λ_{f}, (e.g., the “free” distance between the one or more elements) is on the sub-micro-, micro-, or meso-scale or greater, and (d) the surface of the elements is wettable by the dissolution fluid. Thus if the above conditions are satisfied, an element 430 in the three dimensional structural network may be surrounded by the dissolution fluid 410 soon (e.g. in less than about a minute) after immersion of the dosage form 400. It is assumed that this is the case in the non-limiting example described here. The time to percolate part or all of the free spaces 420 is thus not considered to be rate-determining in dosage form disintegration or drug release.

Subsequent to fluid 410 percolation to the interior of the drug-containing solid 404, the dissolution fluid 410 that surrounds a segment then penetrates into it by diffusion, and the segment may swell and erode. Upon inter-diffusion of the fluid 410 and the polymeric segment, polymer molecules 440 (and gel-layer 450) may spread out. They may intersect with the molecules of adjoining segments at a certain time, t₁, after immersion. Then at t₂ a polymer-fluid solution 460 is formed. The time t₂ to convert the drug-containing solid 404 to such a solution 460 may be estimated by the penetration and erosion times of a single element (or a single segment) 430 in a stagnant fluid 410 (e.g. by Eq. (3)).

If all the free spaces 420 are percolated by the dissolution fluid 410, the concentration of the excipient polymer, c_{e,sol}, in the solution 460 is about:

$${c}_{e , sol} = \frac{{M}_{e}}{{V}_{e} + {V}_{fs}} \frac{{V}_{0}}{{V}_{sol}} = \frac{{φ}_{s} {φ}_{e} {ρ}_{e}}{1 - {φ}_{s} ( 1 - {φ}_{e} )} \frac{{V}_{0}}{{V}_{sol}} \qquad ( 6 )$$

where Mₑ is the mass and Vₑ the volume of the absorptive/soluble excipient, V_{fs} the volume of the free spaces 420, V₀ the initial volume of the dry dosage form, Vₛₒₗ the volume of the solution, φₛ the volume fraction of the solid/dry elements in the dry dosage form, φₑ the volume fraction of the absorptive/soluble excipient polymer in the dry elements 430, and ρₑ is the density of the excipient in the dry state.

The solution 460 is dilute and the polymer molecules disentangled if the excipient concentration in the solution 460, c_{e,sol}≤cₑ*, the disentanglement concentration. This is the case if:

$${φ}_{s} \leq \frac{{V}_{sol} {c}_{e}^{*}}{{V}_{sol} ( 1 - {φ}_{e} ) {c}_{e}^{*} + {V}_{0} {φ}_{e} {ρ}_{e}} \qquad ( 7 )$$

Thus if Eq. (7) is satisfied, the polymer concentration in, or the viscosity of, the solution 460 is so small that the solution 460 is dilute or almost dilute. Consequently, the dosage form can be considered disintegrated as soon the single elements (or segments) 430 are eroded or penetrated. Dosage form 400 disintegration is determined solely by the behavior of a single element 430, and the interactions between elements may be neglected. Thus for an element 430 geometry and properties of the composition as in the non-limiting examples a and b above, the dosage form 400 is disintegrated just a few minutes after immersion. This is well within immediate-release specification, which is one of the most relevant requirements of a typical pharmaceutical dosage form 400.

If the concentration of polymer in the solution 460, c_{e,sol}>>cₑ*, however, the solution 460 may be considered a viscous mass. The viscous mass (or the viscous solution, or the viscous dosage form) then erodes from its exterior surface by diffusion. Thus if the concentration of polymer in (and the viscosity of) the solution 460 are too high, the drug release rate of the dosage form may be reduced substantially. This is detrimental to an immediate-release dosage form. In some embodiments of the invention herein, therefore, the viscosity of the solution 460 formed after inter-diffusion of dissolution fluid 410 and elements 430 is no greater than about 500 Pa·s.

Any more models or examples of the disintegration of a fibrous dosage form in a stagnant fluid obvious to a person of ordinary skill in the art are all within the scope of this invention.

### (f) Dosage Form Disintegration in a Stirred Medium

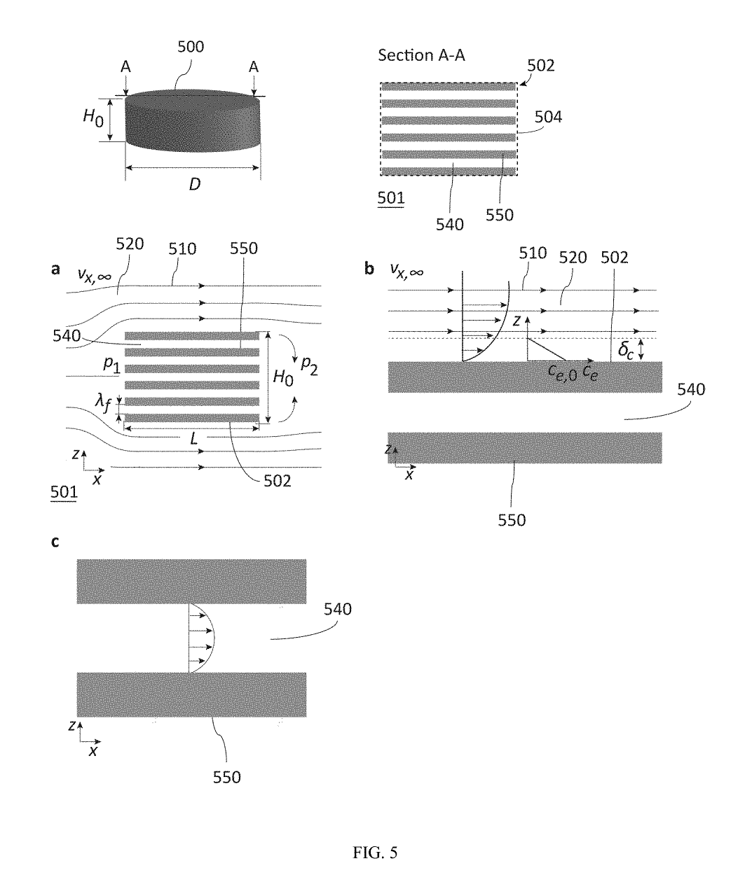

FIG. 5 presents a non-limiting example of dosage form disintegration in a stirred medium. The dosage form 500 comprises a drug-containing solid 501 having an outer surface 502 and an internal structure 504 contiguous with and terminating at said outer surface 502. The outer surface 502 may comprise a solid, or a liquid, or a gas, and is defined as the plane spanned by the structural elements 550 (or segments) at the surface 502 of the drug-containing solid 501. The internal structure 504 comprises a three dimensional structural framework of elements 550. The elements 550 contain an active ingredient and a water-soluble polymeric excipient. The elements 550 further comprise segments separated and spaced from adjoining segments by free spacings, λ_{f}, which define one or more free spaces 540 in the drug-containing solid 501.

Upon immersion in a stirred fluid with far-field velocity, v_{x,∞}, streamlines 510 develop around the dosage form 500 as shown schematically in FIG. 5a. The fluid velocity near the surface 502 is far greater than that in the interior 540. As a result, the erosion rate is greatest at the surface 502. For a case as shown schematically in FIG. 5b the erosion rate of the surface 502 may be approximated by Eq. (4). Using the same parameter values as in section d above, if 10 elements are to be eroded sequentially, the time to erode 80 percent of a dosage form 500 is: t_{dis}≈10×3.7=37 min. This is, however, longer than the required disintegration time of a typical immediate-release dosage form.

Unlike the sequential layer-by-layer removal of material from the surface 502, material removal in the interior 540 of the dosage form is a parallel process because all the elements 550 (e.g. the elements of the internal structure) erode simultaneously. For a velocity profile in the free spaces (or pores) as shown in FIG. 5c, the average fluid velocity in the free spaces, v̅ₓ, may be approximated by:

$${\overline{v}}_{x} = \frac{1}{3} \frac{Δ p {λ}_{f}^{2}}{{μ}_{l} L} \qquad ( 8 )$$

where Δp is the pressure drop across the channel (or across the dosage form), λ_{f} the free spacing between the elements, μₗ the viscosity of the liquid dissolution fluid, and L the channel length.

The pressure drop across the dosage form 500 may be estimated from fluid flow outside the dosage form 500 as:

Δp≈0.5ρₗv_{x,∞}² (9)

Thus the average velocity of the fluid through the internal structure, v̅ₓ, may be estimated as:

$${\overline{v}}_{x} \approx \frac{1}{6} \frac{{ρ}_{l} {v}_{x , ∞}^{2} {λ}_{f}^{2}}{{μ}_{l} L} \qquad ( 10 )$$

For the non-limiting values v_{x,∞}=20 mm/s, ρₗ=1000 kg/m³, μₗ=0.001 Pa·s, λ_{f}=500 μm, L=10 mm, v̅ₓ=1.7 mm/s. x̅ₓ is about 12 times smaller than the far-field velocity in this case.

The erosion rate of an element by convection may be estimated by:

$$\frac{1}{2} \frac{dh}{dt} = - \frac{{j}_{e}}{{ρ}_{e}} \approx \frac{{D}_{e} {c}_{e , 0}}{{ρ}_{e} {\overline{δ}}_{c}} \qquad ( 11 a )$$

where the average concentration boundary layer thickness,

$${\overline{δ}}_{c} \approx 1.56 {( \frac{{D}_{e} {μ}_{l} {L}^{2}}{{ρ}_{l} {v}_{x , ∞}^{2} {λ}_{f}} )}^{1 / 3} \qquad ( 11 b )$$

Thus the erosion time,

$${t}_{E} = \frac{{h}_{0}}{dh / dt} \approx \frac{{h}_{0} {ρ}_{e}}{{D}_{e} {c}_{e , 0}} {( \frac{{D}_{e} {μ}_{l} {L}^{2}}{{ρ}_{l} {v}_{x , ∞}^{2} {λ}_{f}} )}^{1 / 3} \qquad ( 12 )$$

Using the non-limiting values c_{e,0}=163 kg/m³, ρₑ=1150 kg/m³, Dₑ=1.09×10⁻¹⁰ m²/s, ρₗ=1000 kg/m³, μₗ=0.001 Pa·s, v_{x,∞}=20 mm/s, and L=10 mm, the time to erode 80 percent of an element, 0.8×t_{E}=8.3 min.

The calculated t_{E} value is well within immediate-release specification, and shorter than the time to disintegrate the dosage form from the exterior surfaces. Thus, even though the velocity through the internal structure 504 is reduced substantially, material removal by simultaneous erosion of elements 550 in the interior may be faster than by sequential erosion from the surface.

It may be noted, however, that even in a stirred medium, if swelling of fibers in the interior is faster than erosion, the fibrous dosage form may disintegrate as described in the non-limiting example e above. In this case, if expansion of the fibrous structure is unconstrained, the disintegration time of the structure is of the order of the penetration time, tₚₑₙ, of a single fiber (see, e.g., Eq. (3)). But if expansion of the structure is constrained, the dosage form structure may form a “viscous mass” after element swelling (for further details, see, e.g., the non-limiting examples (c) and (e) introduced above). Erosion of such a viscous mass would be mostly from the outer surface, which yields a much longer disintegration time than the simultaneous erosion of elements 550 with appreciable fluid flow through the internal structure 504.

Further details related to convective mass transfer models are given, e.g., in R. B. Bird, W. E. Stewart, E. N. Lightfoot, “Transport phenomena”, 2^{nd} edn., John Wiley & Sons, 2002; and L. Rosenhead, “Laminar boundary layers”, Oxford University Press, 1963. Any more models or examples of the disintegration of a fibrous dosage form in a stirred fluid obvious to a person of ordinary skill in the art are all within the scope and spirit of this invention.

### (g) Summary of Disintegration Models

The above non-limiting models illustrate the effects of the following design parameters on the disintegration rate of single elements and dosage forms: the geometry of the three dimensional structural framework, the solubility of the excipient in the dissolution medium (e.g., the “interfacial concentration” or “critical concentration” or “c_{e,0}”), the diffusivity of the excipient in the dissolution medium, the diffusivity of the medium in the excipient, the fractions of the individual components in the elements, and the disentanglement concentration of the excipient. All these parameters can be deterministically controlled.

Furthermore, the models illustrate that the disclosed dosage forms can be so designed that the length-scale of the disinegration-rate-determining mass transfer step is decreased from the thickness of the dosage form to the thickness (or half-thickness) of the elements. As a result, the disclosed dosage forms can be designed to deliver drug at least an order of magnitude faster than the corresponding non-porous solid forms.

### Dosage Form Design Features

In view of the theoretical models and considerations above, which are suggestive and approximate rather than exact, the design and embodiments of the dosage forms disclosed herein comprise the following.

The pharmaceutical dosage forms disclosed herein comprise a drug-containing solid having an outer surface and an internal structure contiguous with and terminating at said outer surface. The internal structure comprises a three dimensional structural framework of one or more two-dimensional elements. The the two-dimensional elements comprise at least one active ingredient, and in some cases also at least one excipient. The two-dimensional elements further comprise segments separated and spaced from adjoining segments by free spacings, which define one or more free spaces in the drug-containing solid.

For achieving rapid percolation of dissolution fluid into the free spaces, in some embodiments a “free spacing”, λ_{f}, (e.g., a “free” distance between adjoining (i.e., neighboring) elements or adjoining segments) is such that the percolation time of physiological/body fluid into one or more interconnected free spaces of the dosage form is no greater than 900 seconds under physiological conditions. This includes, but is not limited to percolation times no greater than 700 seconds, no greater than 500 seconds, no greater than 300 seconds, no greater than 100 seconds, no greater than 50 seconds, or no greater than 10 seconds, or no greater than 5 seconds under physiological conditions. The pressure of the physiological/body fluid at different positions of the interconnected free spaces may assume different values during fluid percolation.

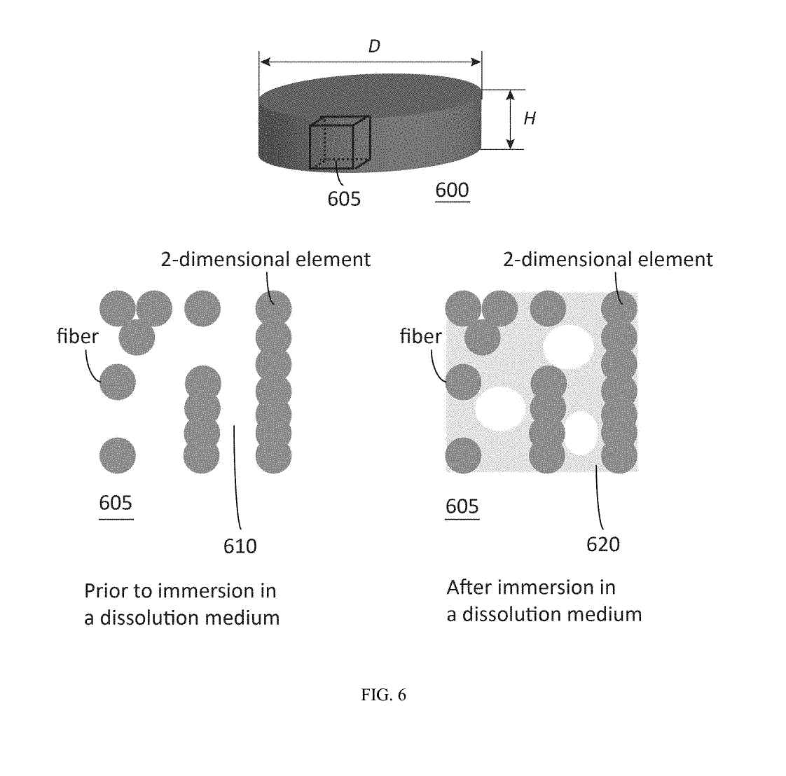

By way of example but not by way of limitation, the percolation time into one or more interconnected free spaces of the dosage form may be determined as follows (FIG. 6). First a volume 605 of the dosage form 600 may be identified that contains one or more interconnected free spaces 610. Then the volume of the interconnected free spaces 610 in said volume of the dosage form 605 may be determined. Then said volume of the dosage form 605 may be immersed in a dissolution medium. Then the volume of dissolution medium 620 that percolated into the volume of the interconnected free spaces 610 of said volume of the dosage form 605 may be determined. As soon as the volume of dissolution medium 620 that percolated into the volume of the interconnected free spaces 610 of said volume of the dosage form 605 is greater than 20 percent of the initial volume of the interconnected free spaces 610, the volume of the interconnected free spaces 610 of said volume of the dosage form 605 may be considered percolated.

Also, in some embodiments, the effective free spacing, λ_{f,e}, on average is greater than 0.1 μm. This includes, but is not limited to an average λ_{f,e} greater than 0.25 μm, or greater than 0.5 μm, or greater than 1 μm, or greater than 2 μm, or greater than 5 μm, or greater than 7 μm, or greater than 10 μm, or greater than 15 μm, or greater than 20 μm, or greater than 25 μm, or greater than 30 μm, or greater than 40 μm, or greater than 50 μm, or in the ranges of 0.1 μm-5 mm, 0.1 μm-3 mm, 0.25 μm-5 mm, 0.5 μm-5 mm, 0.25 μm-3 mm, 0.1 μm-2.5 mm, 0.25 μm-2 mm, 1 μm-4 mm, 5 μm-4 mm, 10 μm-4 mm, 15 μm-4 mm, 20 μm-4 mm, 30 μm-4 mm, 40 μm-4 mm, 50 μm-4 mm, or 1 μm-2 mm. The “effective free spacing” between adjoining segments is defined as the maximum diameter of a sphere that fits in the corresponding free space considering the elements as rigid, fixed bodies. The diameter of such spheres may be estimated from 2-d images of the microstructure. Such 2-d images may be obtained from scanning electron micrographs of the cross section of the dosage form. The greatest circles that fit in the free spaces of the microstructure may be drawn on the scanning electron micrograph (e.g., the 2-d image) and the area-based average diameter of the circles (e.g., the average effective free spacing) may be calculated.

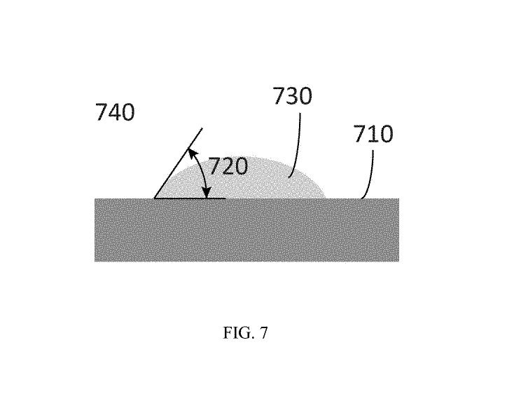

Furthermore, in some embodiments at least one of the one or more excipients is wettable by a physiological/body fluid under physiological conditions. In the context of this work, a solid surface 710 is wettable by a fluid if the contact angle 720 of a fluid droplet 730 on the solid surface 710 exposed to air 740 is no more than 90 degrees (FIG. 7). In some embodiments, the contact angle may not be stationary. In this case, in the invention herein a solid surface is wettable by a fluid if the contact angle 720 of a fluid droplet 730 on the solid surface 710 exposed to air 740 is no more than 90 degrees at least 30-500 seconds after the droplet 730 has been deposited on the surface.

If the two-dimensional elements are parallel to each other, the free spaces between the elements or segments are intrinsically connected to the outer surface of the dosage form. But if some segments or two-dimensional elements are curved or arranged at an angle to each other, closed cells defining one or more free spaces within the three dimensional structural framework of elements may exist. In a closed individual cell or a closed cluster of cells, the free space is entirely surrounded (i.e., enclosed) by solid walls. In some embodiments, a solid wall, or a fraction thereof, is defined by at least one segment of a two-dimensional drug-containing element.

In some embodiments disclosed herein, the following holds. An interconnected, continuous cluster of free space that extends from the outer surface of the drug-containing solid to a given point in the internal structure is obtained if no more than 0 to 12 walls are ruptured (e.g, walls of drug-containing solid enclosing free space are opened or removed). This includes, but is not limited to 0-11, 0-10, 0-9, 0-8, 0-7, 0-6, 0-5, 0-4, or zero walls that must be ruptured to obtain an interconnected cluster of free space that extends from the outer surface to a given point in the internal structure. In FIG. 8, a 2-d example without limitation 800 is presented that shows 3 walls 810 to be ruptured for obtaining an interconnected cluster of free space 820 from point A to point B. For achieving rapid release of drug, the free space of the dosage form is preferably connected to the outer surface. In this case, zero walls must be ruptured to obtain an interconnected cluster of free space that extends from the outer surface to a given point in the internal structure.

For achieving a specific surface area (i.e., surface area-to-volume ratio) large enough to guarantee rapid disintegration of an element, in some embodiments the one or more two-dimensional structural elements have an average thickness, h₀, no greater than 2.5 mm. This includes, but is not limited to h₀ no greater than 2 mm, or no greater than 1.5 mm. It may be noted, however, that if the one or more elements are very thin and thightly packed, the spacing between the segments and elements can be very small, too. This may limit the rate at which dissolution fluid can percolate into or flow through the internal structure upon immersion in a dissolution fluid. Thus, in some embodiments the one or more two-dimensional elements have an average thickness, h₀, in the ranges of 0.1 μm-2.5 mm, 0.5 μm-2.5 mm, 1 μm-2.5 mm, 1.75 μm-2.5 mm, 2.5 μm-2.5 mm, 2.5 μm-2 mm, 5 μm-2 mm, 10 μm-2 mm, 15 μm-2.5 mm, 20 μm-2.5 mm, 30 μm-2.5 mm, or 40 μm-2.5 mm. We may further note that the average thickness of the two-dimensional elements, h₀, can be greater than 2.5 mm in dosage forms that release drug over longer periods of time (e.g., in a time greater than about 25-45 minutes).

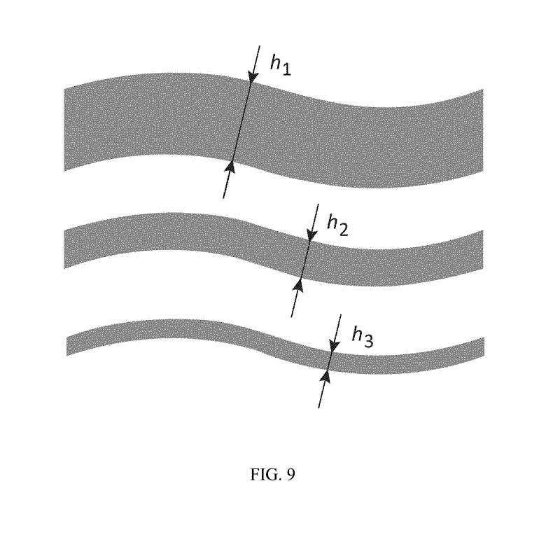

The thickness of a two-dimensional element, h, may be considered the smallest dimension of said element (i.e., h≤w and h≤l, where h, w and l are the thickness, width and length of the element, respectively). The average thickness, h₀, is the average of the thickness along the length and width of the one or more two-dimensional elements in the internal structure. By way of example but not by way of limitation, FIG. 9 presents three elements of equal length and width but of different thicknesses. In this non-limiting example, the average thickness, h₀=(h₁+h₂+h₃)/3. Both the average thickness, h₀, and the thickness of a specific element at a specific position, h, may, for example, be derived from scanning electron micrographs of the cross section of the dosage form.

Moreover, if the dosage form further comprises one or more 0-dimensional or 1-dimensional structural elements, the average thickness of the 0D-elements or 1D-elements may be no greater than 2.5 mm in some embodiments disclosed herein. By way of example but not by way of limitation, this includes an average thickness of 0D-elements or 1D-elements no greater than 2 mm, or in the ranges of 0.1 μm-2.5 mm, 0.25 μm-2.5 mm, 0.5 μm-2.5 mm, 2 μm-2.5 mm, 2.5 μm-2 mm, 1 μm-2 mm, 0.5 μm-1.5 mm, or 2 μm-2 mm. The average thickness of a one-dimensional structural element is referred to as the average of the thickness along the length of the element. The average thickness of a zero-dimensional structural element is referred to as the thickness of the element (e.g., the smallest dimension of the element).

Also, we may note that the cross section of a 2D-element (and the cross sections of a 0D-element or 1D-element, too) may assume any shape. Thus, by way of example but not by way of limitation, the cross section may be polygonal, ellipsoidal, circular, rectangular, combinations thereof, and so on. Furthermore, the cross section of a 2D, 1D, or 0D-element may vary along the length of said element.

A two-dimensional element or a segment in the three dimensional structural framework of one or more two-dimensional elements may, for example, be defined by its position (e.g., the position of its center of mass, the central plane of the element, etc.) relative to a reference point or frame. (In the invention herein, a reference frame may be understood as a reference coordinate system.) The reference point or the origin and orientation of the reference frame may be specified on the outer surface or within the internal structure of the drug containing solid.

In some embodiments of the invention herein, the position of at least one two-dimensional element or at least one segment in the internal structure is precisely controlled. Such embodiments include, but are not limited to internal structures wherein the position of a fraction of the elements or segments that make up the three dimensional structural framework of one or more elements is precisely controlled. The volume fraction of elements or segments (with respect to the total volume of elements or segments that make up the three dimensional structural framework of one or more elements) of which the position is precisely controlled can be greater than 0.1, or greater than 0.3, or greater than 0.5, or greater than 0.7, or greater than 0.9. It may be noted that in the context of the invention herein, a variable or a parameter (e.g., the position of an element, or a free spacing, or a thickness of an element) is precisely controlled if it is deterministic and not stochastic (or random). A variable or parameter may be deterministic if, upon multiple repetitions of a step that includes said variable, the standard deviation of the values of said variable is smaller than the average value. This includes, but is not limited to a standard deviation of the values of said variable smaller than half the average value, or smaller than one third of the average value, or smaller than a quarter of the average value, or smaller than one fifth or the average value, or smaller than one sixth of the average value of said variable.

In some embodiments, furthermore, at least one spacing between segments, λ, and/or at least one free spacing between segments, and/or at least one thickness of a segment or element, h, is/are precisely (or deterministically) controlled. Thus, in some embodiments herein, if an element or segment is produced multiple times under identical conditions, the standard deviation of the thickness of said element or segment is less than the average value of said element's thickness. Similarly, if an inter-element or inter-segment spacing is produced multiple times under identical conditions, the standard deviation of said inter-element or inter-segment spacing is less than the average value in certain embodiments of the invention herein. It may be noted that the inter-element or inter-segment spacing may change along the length or width of said element or segment. Similarly, also the thickness of an element or a segment may change along its length or width.

A non-limiting example of a three dimensional structural framework of one or more elements wherein the position of a large fraction (or all) of the elements, the inter-element spacing, and the element thickness are controlled (or precisely controlled) is an ordered structure. Non-limiting schematics of ordered structures are shown in FIGS. 1a-1e and FIG. 2. The advantage of ordered structures over disordered or random structures is that the microstructure (e.g., the geometry of the free spaces, etc.) and the properties (e.g., the drug release rate by the structure) can be better controlled.

Typically, the volume fraction of two-dimensional structural elements in the dosage form is no greater than 0.98. In other non-limiting examples, the volume fraction of elements in the dosage form is no greater than 0.95, or no greater than 0.93, or no greater than 0.9. In most cases, it is in the range 0.1-0.9, depending on how the one or more elements are arranged. A small volume fraction of elements is desirable to fill small amounts of drug in a comparable large volume (e.g., if the dosage form is used for delivery of a highly potent drug with a drug dose of just a few milligrams or less). On the contrary, a large volume fraction of elements is desirable to fill large amounts of drug in a small volume (e.g., if the dosage form is used for delivery of a low potency drug or delivery of multiple active ingredients with a total drug dose of several 100 mg or more).

For achieving rapid erosion of elements after contact with physiological/body fluids, in some embodiments the two-dimensional elements include at least one excipient that has a solubility greater than 0.1 g/l in physiological/body fluids under physiological conditions. This includes, but is not limited to a solubility by at least one excipient in a physiological/body fluid greater than 0.5 g/l, or greater than 1 g/l, or greater than 5 g/l, or greater than 10 g/l, or greater than 20 g/l, or greater than 30 g/l, or greater than 50 g/l, or greater than 70 g/l, or greater than 100 g/l. Furthermore, the diffusivity of a dissolved excipient molecule in a physiological/body fluid may be greater than 1×10⁻¹² m²/s under physiological conditions. This includes, but is not limited to a diffusivity of a dissolved excipient molecule in a physiological/body fluid greater than 2×10⁻¹² m²/s, greater than 4×10⁻¹² m²/s, greater than 6×10⁻¹² m²/s, greater than 8×10⁻¹² m²/s, or greater than 1×10⁻¹¹ m²/s under physiological conditions. The volume fraction of soluble excipient in the excipient (e.g., the excipient in its totality or all the volume of the one or more excipients in the one or more fibers) may be greater than 0.02. This includes, but is not limited to volume fractions of the soluble excipient in the excipient greater than 0.04, greater than 0.06, greater than 0.08, or greater than 0.1.

In polymers that form viscous solutions when combined with a dissolution medium, the ‘solubility’ in the context of this invention is the polymer concentration in physiological/body fluid at which the average shear viscosity of the polymer-physiological/body fluid solution is 5 Pa·s in the shear rate range 1-100 l/s under physiological conditions. The pH value of the physiological/body fluid may thereby be adjusted to the specific physiological condition of interest. By contrast, the solubility of a material that does not form a viscous solution when combined with a dissolution medium is the maximum amount of said material dissolved in a given volume of dissolution medium at equilibrium divided by said volume of the medium. It may, for example, be determined by optical methods.

Furthermore, in some embodiments the one or more elements include at least one excipient that is absorptive of a physiological/body fluid. The effective diffusivity of physiological/body fluid in an absorptive excipient (and/or an element) may be greater than 0.5×10⁻¹¹ m²/s under physiological conditions. In other examples without limitation, the effective diffusivity of physiological/body fluid in an absorptive excipient (and/or an element) may be greater than 1×10⁻¹¹ m²/s, greater than 3×10⁻¹¹ m²/s, greater than 6×10⁻¹¹ m²/s, or greater than 8×10¹¹ m²/s under physiological conditions.

Alternatively, (e.g., for absorptive excipients where diffusion of physiological/body fluid to the interior is not Fickian) a rate of penetration may be specified. In some embodiments, the rate of penetration of a physiological/body fluid into a solid, absorptive excipient (and/or an element) is greater than an average thickness of the one or more elements in the internal structure divided by 3600 seconds (i.e., h₀/3600 μm/s). In other examples without limitation, rate of penetration may be greater than h₀/1800 μm/s, greater than h₀/1200 μm/s, greater than h₀/800 μm/s, or greater than h₀/600 μm/s.

For determining the effective diffusivity (and/or the rate of penetration) of dissolution medium in a solid, absorptive excipient (and/or an element) the following procedure may be applied. An element (e.g an element of the dosage form structure or an element that just consists of the absorptive excipient) may be fixed at two ends and placed in a still dissolution medium at 37° C. The time t₁ for the element to break apart or deform substantially may be recorded. (By way of example but not by way of limitation, a deformation of an element may be considered substantial if either the length, width, or thickness of the element differs by more than 10 to 20 percent from its initial value. In elements with weight fraction, wₑ, or volume fraction, φₑ, of absorptive/swellable excipient smaller than 0.4, a deformation of an element may be considered substantial if either the length, width, or thickness of the element differs by more than 25×φₑ percent or 25×wₑ percent from its initial value.) The effective diffusivity, D_{eff}, may then be determined according to D_{eff}=h₀²/4t₁ where h₀ is the initial element thickness (e.g., the thickness of the dry element). Similarly, the rate of penetration of a physiological/body fluid into the element is equal to h₀/2t₁.

The effective diffusivity of dissolution medium in or the average velocity at which the fluid front advances (i.e., the rate of penetration of a physiological/body fluid) into a solid, absorptive excipient (or an element) may also be determined by spectral methods. By way of example but not by way of limitation, one side of an element may be exposed to the dissolution medium. On the other side of the element, the concentration of dissolution medium may be monitored. As soon as the monitored concentration of dissolution medium raises substantially (e.g., as soon as the concentration of water or dissolution fluid in the absorptive/swellable excipient on the monitored surface is greater than twice the concentration of water or dissolution fluid in the absorptive/swellable excipient of the initial solid element), the element is penetrated. The time t₁ to penetrate the element may be recorded and the effective diffusivity and rate of penetration calculated as detailed in the previous paragraph. Spectral methods are suited for materials that have some mechanical strength (i.e., increased viscosity) when they are penetrated by the dissolution fluid. They are also suited for materials (or elements) where the deformation of the element upon penetration of dissolution fluid is small.

In some embodiments, at least one excipient of the drug-containing solid transitions from solid to a fluidic or gel consistency solution upon being solvated with a volume of physiological/body fluid equal to the volume of the one or more free spaces of the drug-containing solid (or dosage form). To ensure that the disintegration rate of such a drug-containing solid is of the order of the disintegration rate of a single element (e.g., to avoid that the drug-containing solid forms a viscous mass upon immersion in a dissolution medium that erodes slowly from its outer surfaces), the viscosity of said solution is no greater than 500 Pa·s. In other words, a solution comprising the weight of soluble/absorptive excipient in the drug-containing solid and a volume of physiological/body fluid equal to the volume of the free spaces of the drug-containing solid (specifically the volume of the free spaces that are removable by the dissolution fluid), has a viscosity no greater than 500 Pa·s. This includes, but is not limited to a viscosity of said solution less than 400 Pa·s, less than 300 Pa·s, less than 200 Pa·s, less than 100 Pa·s, less than 50 Pa·s, less than 25 Pa·s, or less than 10 Pa·s. In the context of this work, the viscosity of a solution is the average shear viscosity of the solution in the shear rate range 1-100 l/s under physiological conditions.

Non-limiting examples of excipients that if used at the right quantities satisfy some or all of the above requirements include polyethylene glycol (PEG), polyvinylpyrrolidone (PVP), PEG-PVP copolymer, poloxamer, lauroyl macrogol-32 glycerides, polyvinylalcohol (PVA), PEG-PVA copolymer, polylactic acid, polyvinylacetate phthalate, polymethacrylates (e.g., poly(methacrylic acid, ethyl acrylate) 1:1, butylmethacrylat-(2-dimethylaminoethyl)methacrylat-methylmathacrylat-copolymer), gelatin, cellulose or cellulose derivatives (e.g., microcrystalline cellulose, hydroxypropyl cellulose, hydroxyethyl cellulose, methyl cellulose, hydroxypropyl methyl ether cellulose, hydroxypropyl methylcellulose), starch, polylactide-co-glycolide, polyvinyl caprolactam-polyvinyl acetate-polyethylene glycol graft copolymer, pregelatinized starch, lactose, sodium starch glycolate, polyacrylic acid, acrylic acid crosslinked with allyl sucrose or allyl pentaerythritol (e.g., carbopol), or polyols (e.g., lactitol, maltitol, mannitol, isomalt, xylitol, sorbitol, maltodextrin, etc.), among others.

In some embodiments, the average molecular weight of an excipient may be in the range 1,000 g/mol to 300,000 g/mol. This includes, but is not limited to an average molecular weight of the excipient in the range 2,000 g/mol to 200,000 g/mol.

The one or more free spaces may be filled with a matter selected from the group comprising solid, liquid, gas (or vacuum), or combinations thereof. If one or more elements (or one or more segments) is/are partially or entirely surrounded by free space, the content of said free space may be removed partially or entirely after contact with dissolution fluid to give the fluid access to the elements. This condition is, for example, satisfied by gases. Examples of biocompatible gases that may fill the free space include air, nitrogen, CO₂, argon, oxygen, and nitric oxide, among others.

Liquids that are partially or entirely removed from the structure upon contact with dissolution fluid, and thus may be used to fill the free spaces include, but are not limited to such biocompatible low viscosity fluids as: Polyethylene glycol (PEG) with molecular weight smaller than about 1000 Da (e.g. PEG 400, PEG 300, etc.), Poloxamer 124, 2-Pyrrolidone, Glycerol triacetate (Triacetin), D-alpha tocopheryl polyethylene glycol 1000 succinate (TPGS), Polyoxyl Hydroxystearate, Polyoxyl 15 Hydroxystearate, Castor oil, Polyoxyl castor oil (Polyethoxylated castor oil), Polyoxyl 35 castor oil, Polyoxyl hydrogenated castor oil, Glyceryl monooeleate, Glycerin, Propylene glycol, Propylene carbonate, Propionic acid, Peanut oil, water, Sesame oil, Olive oil, Almond oil, combinations of such (and/or other) liquids with a polymer or any other molecule that dissolves in them, among others.

Non-limiting examples of solids that are removed or dissolved after contact with physiological/body fluid include sugars or polyols, such as Sucrose, Lactose, Maltose, Glucose, Maltodextrin, Mannitol, Maltitol, Isomalt, Lactitol, Xylitol, Sorbitol, among others. Other examples of solids include polymers, such as polyethylene glycol, polyvinyl pyrrolidone, polyvinyl alcohol, among others. Other examples of solids include effervescent agents, such as sodium bicarbonate. The relevant physical properties of a solid that is bonded to a drug-containing fiber are high solubility and diffusivity in physiological/body fluids to ensure its rapid removal after contact with physiological/body fluid. Thus other non-limiting examples of a solid include solid active pharmaceutical ingredients with high solubility and diffusivity, such as Aliskiren. Typically, a solid material should have a solubility in physiological/body fluid under physiological conditions greater than 50 g/l to be removed or dissolved rapidly after contact with dissolution medium. This includes, but is not limited to a solubility greater than 75 g/l, or greater than 100 g/l, or greater than 150 g/l. The diffusivity of the solid material (as dissolved molecule in physiological/body fluid under physiological conditions) should typically be greater than 4×10⁻¹² m²/s if the solid material must be dissolved rapidly after contact with dissolution medium. This includes, but is not limited to a diffusivity greater than 6×10⁻¹² m²/s, or greater than 8×10⁻¹² m²/s, or greater than 1×10⁻¹¹ m²/s.

Furthermore, one or more filler materials such as microcrystalline cellulose or others, one or more sweeteners, one or more taste masking agents, one or more stabilizing agents, one or more preservatives, one or more coloring agents, or any other common or uncommon excipient may be added as excipient to the dosage form.

In some embodiments, a disintegration time of the dosage form (or the drug-containing solid) is no greater than 50 minutes. This includes, but is not limited to a disintegration time no greater than 40 minutes, no greater than 30 minutes, no greater than 25 minutes, no greater than 20 minutes, or no greater than 15 minutes. In the context of this disclosure, the disintegration time is defined as the time required to release 80 percent of the drug content of a representative dosage form structure into a stirred dissolution medium. The released drug may be a solid, such as a solid drug particle, and/or a molecule, such as a dissolved drug molecule. The disintegration test may, for example, be conducted with a USP disintegration apparatus under physiological conditions. (See, e.g. The United States Pharmacopeial Convention, USP 39-NF 34). Another method without limitation to conduct a disintegration test is by a USP basket apparatus (i.e., a USP apparatus 1 as shown in The United States Pharmacopeial Convention, USP 39-NF 34) under physiological conditions (e.g., at a temperature of 37° C. and at a stirring rate or basket rotation rate of 50-150 rpm). In this method, the time to disintegrate 80 percent of the representative dosage form structure after immersion in the stirred dissolution medium may, for example, be determined by visual or other optical methods. It may be noted that if the drug is in molecular form immediately or almost immediately after it is released from the dosage form structure, the disintegration time is about the same as the time to dissolve 80% of the drug content of a representative dosage form structure after immersion in a stirred dissolution medium.

In case the elements are well bonded to each other (or to a solid material that fills the one or more free spaces), the greater of a tensile strength or a yield strength of the assembled dosage form material (e.g., the dosage form or the drug-containing solid) is no less than 0.005 MPa. In other examples without limitation, the greater of a tensile strength or a yield strength of the assembled dosage form material is no less than 0.01 MPa, or 0.015 MPa, or 0.02 MPa, or 0.025 MPa, or 0.04 MPa, or 0.06 MPa, or 0.1 MPa, or 0.25 MPa, or 0.5 MPa.

In some embodiments, the dosage form may be coated. A coating may serve as taste masking agent, protective coating, means of providing color to the dosage form, enteric coating, means of improving the aesthetics of the dosage form, or have any other common or uncommon function of a coating. Moreover, in some non-limiting examples of the invention herein, a coating may be applied on the 2D-elements of the three dimensional structural framework of one ore more 2D-elements.

Also the coating materials include, but are not limited to polyethylene glycol (PEG), polyvinylpyrrolidone (PVP), PEG-PVP copolymer, poloxamer, lauroyl macrogol-32 glycerides, polyvinyl alcohol (PVA), PEG-PVA copolymer, polylactic acid, polyvinyl acetate phthalate, polymethacrylates (e.g., poly(methacrylic acid, ethyl acrylate) 1:1, butylmethacrylat-(2-dimethylaminoethyl)methacrylat-methylmathacrylat-copolymer), gelatin, cellulose or cellulose derivatives (e.g., microcrystalline cellulose, hydroxypropyl cellulose, hydroxyethyl cellulose, methyl cellulose, hydroxypropyl methyl ether cellulose, hydroxypropyl methylcellulose), starch, polylactide-co-glycolide, polyvinyl caprolactam-polyvinyl acetate-polyethylene glycol graft copolymer, pregelatinized starch, lactose, sodium starch glycolate, or polyacrylic acid, Sucrose, Lactose, Maltose, Glucose, Maltodextrin, Mannitol, Maltitol, Isomalt, Lactitol, Xylitol, Sorbitol, a sweetener, a coloring agent, a preservative, a stabilizer, a taste masking agent, among others.

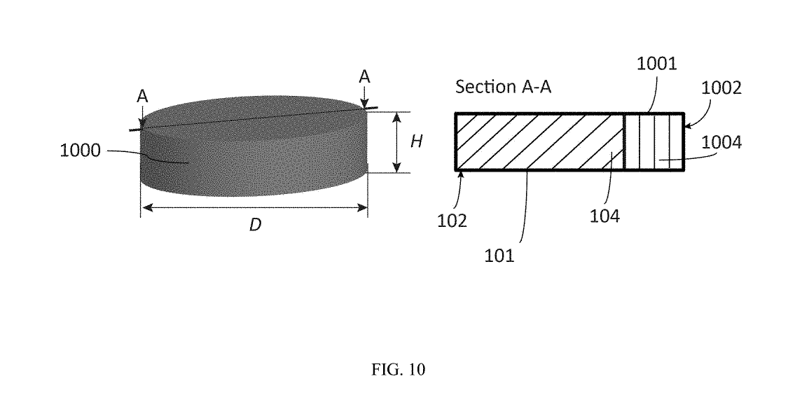

In some embodiments, in addition to the drug-containing solid 101 described above, the dosage form 1000 disclosed herein may comprise another drug-containing solid 1001 that contains at least one active ingredient (or one or more other drug-containing solids that contain at least one active ingredient; all such other drug-containing solids are referred to here as “other solid” or “other drug-containing solid”). Said other drug-containing solid 1001 has an outer surface 1002 and internal structure 1004 contiguous with and terminating at said outer surface 1002 as shown in FIG. 10. In some embodiments, 80 percent of the other solid's 1001 drug content is converted to dissolved molecules in a time greater than 60 minutes after immersion of the dosage form in a physiological/body fluid under physiological conditions. In other embodiments, 80 percent of the other solid's 1001 drug content is converted to dissolved molecules in a time no greater than 60 minutes after immersion of the dosage form in a physiological/body fluid under physiological conditions.

In some embodiments, a two-dimensional elements may comprise multiple layers of different materials. This includes, but is not limited to a coating.

### EXPERIMENTAL EXAMPLES

The following examples illustrate ways by which the dosage forms may be prepared and analyzed, and will enable one of skill in the art to more readily understand the principle thereof. The examples are presented by way of illustration and are not meant to be limiting in any way.

### Example 1: Preparation of Dosage Forms

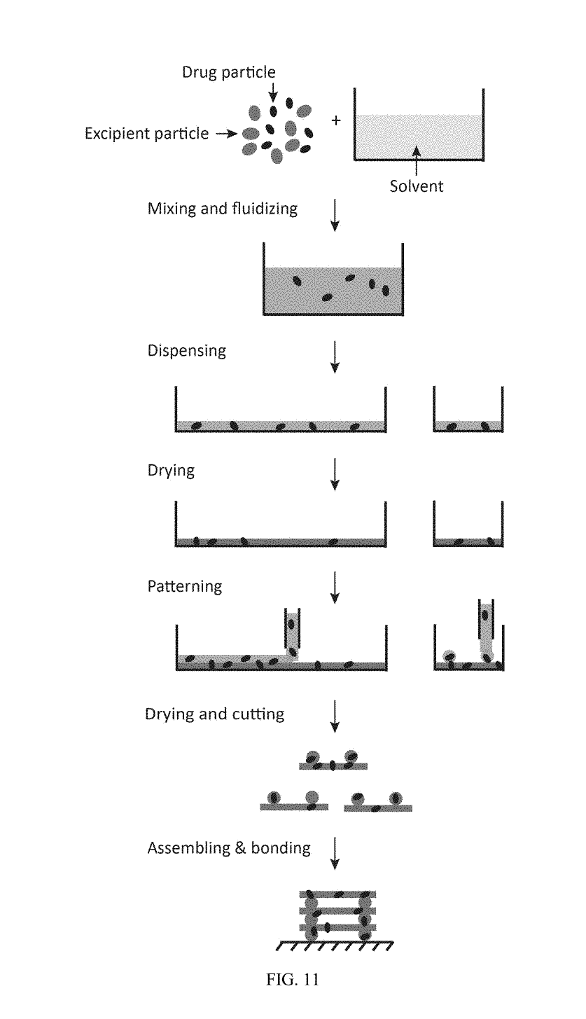

Dosage forms were prepared as shown schematically in FIG. 11. Drug (acetaminophen) and excipient (polyvinyl alcohol-polyethylene glycol graft copolymer 3:1 of molecular weight 45 kg/mol) particles were first combined and mixed with a solvent (water) to form a liquid dispersion of dissolved excipient, dissolved drug, solvent, and drug particles. The weight fraction of drug in the liquid dispersion was 0.09, the weight fraction of excipient 0.25, and the weight fraction of water 0.66. About 0.35 ml of the dispersion was then dispensed into an open mold with a width of 10 mm and a length of 100 mm. Subsequently, the dispersion was exposed to an air stream at 60° C. for 15 minutes to evaporate the solvent and form a thin film. A fiber pattern was then deposited on the solid film. The composition of the fibers was 0.14 wt % acetaminophen, 0.38 wt % polyvinyl alcohol-polyethylene glycol graft copolymer 3:1 of molecular weight 45 kg/mol and 0.48 wt % water. The radius of the wet fibers was about 250 μm and the inter-fiber spacing was about 6 mm. Finally, the film was cut into square disks of 10 mm side length; the disks (e.g., the elements) were then assembled, bonded to a dosage form structure, and dried. The dosage forms were square disks: 10 mm in side length and about 5 mm in thickness.

### Example 2: Dosage Form Microstructures

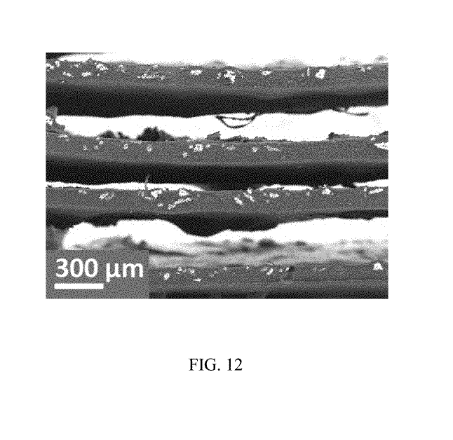

FIG. 12 presents a scanning electron micrograph of the microstructure of a dosage form produced as detailed in the non-limiting experimental example 1. The thickness of the 2D-elements in the dosage form structure was 120±10 μm and the free spacing between the 2D-elements was 285±96 μm.

### Example 3: Drug Release

Drug release by a dosage form that was prepared as detailed in example 1 was tested using a USP dissolution apparatus 1 (as shown, e.g., in The United States Pharmacopeial Convention, USP 39-NF 34). The apparatus was filled with 900 ml of the dissolution fluid (a 0.05 M phosphate buffer solution with pH 5.8 at a temperature of 37±2° C.). The basket was rotated at 50 rpm. The concentration of dissolved drug in the dissolution fluid was measured versus time by UV absorption at 244 nm using a fiber optic probe. For all the dosage forms, the fraction of drug dissolved increased steadily with time at roughly constant rate until it plateaud out to the final value.

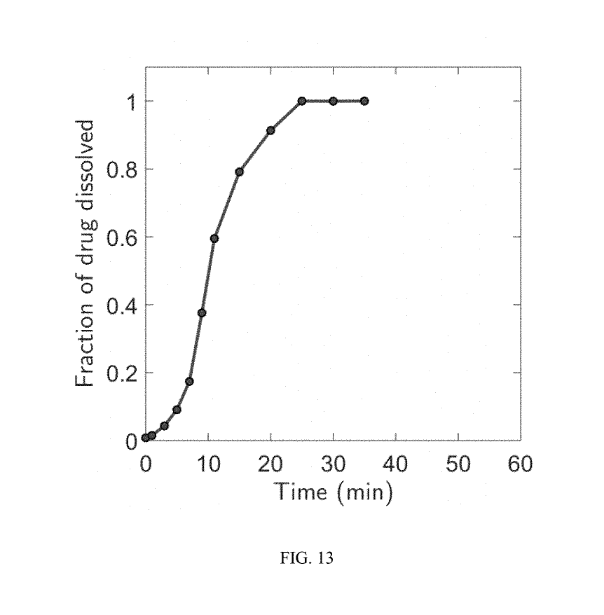

FIG. 13 presents a representative curve of the fraction of drug dissolved versus time of the dosage form prepared as detailed in the non-limiting experimental example 1. The fraction of drug dissolved increased steadily with time and then plateaud out to the final value. The time to dissolve 80% of the drug content, t_{0.8}, could thus be readily extracted: it was 15 minutes.

### Dosage Form Application Examples

In some embodiments, the amount of active ingredient contained in a dosage form disclosed in this invention is appropriate for administration in a therapeutic regimen that shows a statistically significant probability of achieving a predetermined therapeutic effect when administered to a relevant population. By way of example but not by way of limitation, active ingredients may be selected from the group consisting of acetaminophen, aspirin, caffeine, ibuprofen, an analgesic, an anti-inflammatory agent, an anthelmintic, anti-arrhythmic, antibiotic, anticoagulant, antidepressant, antidiabetic, antiepileptic, antihistamine, antihypertensive, antimuscarinic, antimycobacterial, antineoplastic, immunosuppressant, antihyroid, antiviral, anxiolytic and sedatives, beta-adrenoceptor blocking agents, cardiac inotropic agent, corticosteroid, cough suppressant, diuretic, dopaminergic, immunological agent, lipid regulating agent, muscle relaxant, parasympathomimetic, parathyroid, calcitonin and biphosphonates, prostaglandin, radiopharmaceutical, anti-allergic agent, sympathomimetic, thyroid agent, PDE IV inhibitor, CSBP/RK/p38 inhibitor, or a vasodilator).

In conclusion, this invention discloses a dosage form with predictable structure and drug release behavior. Both can be tailored by well-controllable parameters. This enables faster and more economical pharmaceutical development and manufacture, a greater range of dosage form properties, improved quality of the dosage forms, and more personalized medical treatments.

It is contemplated that a particular feature described either individually or as part of an embodiment in this disclosure can be combined with other individually described features, or parts of other embodiments, even if the other features and embodiments make no mention of the particular feature. Thus, the invention herein extends to such specific combinations not already described. Furthermore, the drawings and embodiments of the invention herein have been presented as examples, and not as limitations. Thus, it is to be understood that the invention herein is not limited to these precise embodiments. Other embodiments apparent to those of ordinary skill in the art are within the scope of what is claimed.

We claim:

1. A pharmaceutical dosage form comprising:
a drug-containing solid having an outer surface and an internal structure contiguous with and terminating at said outer surface;
said internal structure comprising a three dimensional structural framework of one or more two-dimensional elements;
said two-dimensional elements comprising at least one active ingredient and at least one excipient;
said two-dimensional elements further comprising segments separated and spaced from adjoining segments by free spacings; and
the free spacings defining one or more free spaces in said drug-containing solid.

2. The dosage form of claim 1, wherein the internal structure further comprises one or more zero-dimensional elements.

3. The dosage form of claim 1, wherein the internal structure further comprises one or more one-dimensional elements.

4. The dosage form of claim 1, wherein the one or more 2-dimensional elements comprise an average thickness no greater than 2.5 mm.

5. The dosage form of claim 1, wherein the free spacing between the segments is so that the percolation time of physiological/body fluid into one or more interconnected free spaces of the dosage form is no greater than 900 seconds under physiological conditions.

6. The dosage form of claim 1, wherein the effective free spacing between segments across the one or more free spaces on average is greater than 0.1 μm.

7. The dosage form of claim 1, wherein the position of at least one two-dimensional element or at least one segment in the internal structure is precisely controlled.

8. The dosage form of claim 1, wherein the three dimensional framework of one or more two-dimensional elements comprises an ordered structure.

9. The dosage form of claim 1, wherein the thickness of at least one two-dimensional element is precisely controlled.

10. The dosage form of claim 1, wherein at least one excipient is wettable by a physiological/body fluid under physiological conditions.

11. The dosage form of claim 1, wherein at least one excipient is soluble in a physiological/body fluid and comprises a solubility greater than 0.1 g/l in said physiological/body fluid under physiological conditions.

12. The dosage form of claim 11, wherein dissolved molecules of the soluble excipient comprise a diffusivity greater than 0.2×10⁻¹² m²/s in a physiological/body fluid under physiological conditions.

13. The dosage form of claim 1, wherein at least one excipient is absorptive of a physiological/body fluid, and wherein rate of penetration of the physiological/body fluid into a two-dimensional element or said absorptive excipient under physiological conditions is greater than the average thickness of said two-dimensional element divided by 3600 seconds.

14. The dosage form of claim 1, wherein at least one excipient is absorptive of a physiological/body fluid, and wherein an effective diffusivity of physiological/body fluid in a two-dimensional element or said absorptive excipient is greater than 0.5×10⁻¹¹ m²/s under physiological conditions.

15. The dosage form of claim 1, wherein at least one excipient transitions from solid to a fluidic or gel consistency solution upon contact with a volume of physiological/body fluid equal to the volume of the one or more free spaces of the drug-containing solid, said solution having a viscosity less than 500 Pa·s under physiological conditions.

16. The dosage form of claim 1, wherein at least one excipient is selected from the group comprising polyethylene glycol (PEG), polyethylene oxide, polyvinylpyrrolidone (PVP), PEG-PVP copolymer, poloxamer, lauroyl macrogol-32 glycerides, polyvinylalcohol (PVA), PEG-PVA copolymer, polylactic acid, polyvinylacetate phthalate, polymethacrylates (e.g., poly(methacrylic acid, ethyl acrylate) 1:1, or butylmethacrylat-(2-dimethylaminoethyl)methacrylat-methylmathacrylat-copolymer), gelatin, cellulose or cellulose derivatives (e.g., microcrystalline cellulose, hydroxypropyl cellulose, hydroxyethyl cellulose, methyl cellulose, hydroxypropyl methyl ether cellulose, or hydroxypropyl methylcellulose), starch, polylactide-co-glycolide, polyvinyl caprolactam-polyvinyl acetate-polyethylene glycol graft copolymer, lactose, starch derivatives (e.g., pregelatinized starch or sodium starch glycolate), chitosan, pectin, polyols (e.g., lactitol, maltitol, mannitol, isomalt), acrylic acid crosslinked with allyl sucrose or allyl pentaerythritol (e.g., carbopol), and polyacrylic acid.

17. The dosage form of claim 1, wherein a free space is filled with a matter selected from the group comprising gas, liquid, or solid, or combinations thereof, and wherein said matter is partially or entirely removed upon contact with a physiological/body fluid under physiological conditions.

18. The dosage form of claim 17, wherein the gas comprises at least one of air, nitrogen, CO₂, argon, or oxygen.

19. The dosage form of claim 1, wherein the free spaces are interconnected.

20. The dosage form of claim 1, wherein less than twelve walls must be ruptured to obtain an interconnected cluster of free space from the outer surface of the drug-containing solid to any point in the internal structure.

21. A pharmaceutical dosage form comprising:
a drug-containing solid having an outer surface and an internal structure contiguous with and terminating at said outer surface;
said internal structure comprising a three dimensional structural framework of one or more two-dimensional elements;
said two-dimensional elements comprising at least one active ingredient and at least one excipient;
said two-dimensional elements further comprising segments separated and spaced from adjoining segments by free spacings; and
the free spacings defining one or more free spaces in said drug-containing solid;
wherein
the one or more two-dimensional elements comprise an average thickness no greater than 2.5 mm;
the effective free spacing between the segments across the one or more free spaces on average is between 0.1 μm and 2 mm; and
at least one dimension of the dosage form is greater than 1 mm.

22. A pharmaceutical dosage form comprising:
a drug-containing solid having an outer surface and an internal structure contiguous with and terminating at said outer surface;
said internal structure comprising a three dimensional structural framework of one or more two-dimensional elements;
said two-dimensional elements comprising at least one active ingredient and at least one excipient;
said two-dimensional elements further comprising segments separated and spaced from adjoining segments by free spacings; and
the free spacings defining one or more free spaces in said drug-containing solid;
wherein
the one or more two-dimensional elements comprise an average thickness no greater than 2.5 mm;
the effective free spacing between the segments across the one or more free spaces on average is between 0.1 μm and 2 mm;
at least one dimension of the dosage form is greater than 1 mm; and
at least one excipient comprises a solubility greater than 0.1 g/l in a physiological/body fluid under physiological conditions or at least one excipient is absorptive of a physiological/body fluid, and wherein rate of penetration of the physiological/body fluid into a two-dimensional element or an absorptive excipient under physiological conditions is greater than average thickness of the two-dimensional elements divided by 3600 seconds.
